(19) United States
(12) Patent Application Publication
(10) Pub. No.: US 2007/0219444 A1
(43) Pub. Date: Sep. 20, 2007

(54) Apparatus and method for guiding catheters

(76) Inventors: Cesar M. Diaz, Rancho Santa Margarita, CA (US); Kim McGurrin, Guelph (CA); Peter Physick-Sheard, Rockwood (CA)

Correspondence Address: Charles D. Gunter, Jr.;Whitaker, Chalk, Swindle & Sawyer, LLP, Suite 3500, 301 Commerce Street, Fort Worth, TX, 76102-4186, US

(21) Appl. No.: 11/602,860

(22) Filed: Nov. 21, 2006

Related U.S. Application Data
(63) Continuation-in-part of application No. 11/363,361, filed on Feb. 27, 2006.
Application No. 11/363,361 is a continuation-in-part of application No. 10/757,948, filed on Jan. 14, 2004, now abandoned.

(51) Int. Cl. A61B 8/00; A61B 5/05

(52) U.S. Cl. 600/424; 600/438; 600/439

(57) ABSTRACT

The present invention is a system for guiding catheters into chamber or conduits of the body without the use of X-ray based imaging systems. The system disclosed is used for guidance of catheters in the heart chamber and heart protruding structures and conduits by using external ultrasound and device based physiological sensory inputs to create a quasi-visual-sensory-algorithm that is used to provide clinical proper sensory and handling input so that device placement is facilitated. The method and preferred devices are designed to deliver high energy defibrillation shocks to the myocardium and also provide a stable substrate for pressure lumens and or sensors used to provide “distal specific” physiological sensory inputs.

### BACKGROUND OF THE INVENTION

1. Field of the Invention

The present invention generally relates to devices and methods used to guide catheters into body lumens and chambers using a set of non-X ray based methods that include ultrasound and pressure gradients to form a quasi-visual-sensory-algorithm that can be used to guide the devices into a desired location within the body.

2. Description of the Prior Art

Atrial Fibrillation is a cardiac disease that has been widely reported in humans throughout the world with patient population estimates ranging in the 6 to 10 million with an annual compound growth rate estimated at 7% per annum. The disease is complex and commonly associated with chaotic electrical disturbances found in the atria but originating from a variety of regions in and around the heart. The disease is further complicated by differences in underlying disease states such as structural heart disease and coronary artery disease. Considerable work has been done and apparatus and methods defined to terminate this disease using internal cardioversion by contributors including Levy, Dolla, Jaros, Alt, Accorti, Diaz, and others.

Most of the prior work concerns devices with utility in the electrophysiology laboratory. The devices for the most part define a sensing catheter that also delivers electrical energy to terminate “shockable” arrhythmias with less energy than an external defibrillator. However, early work done as part of the Rhythm Clinical Study for Cardiac Arrhythmias that was initiated in 1995 showed anecdotal evidence of a curative effect of some atrial fibrillation patients. However, at the time, the data was considered to be too inconsistent and unclear and possibly an artifact. The prevailing concern at the time was energy reduction driven by the desire to create a painless implantable internal cardiac defibrillator (ICD). The ICD science dominated the field's interest and also the minds of many of the researchers perfecting this technology. Work done by Mirowski (U.S. Pat. No. 3,616,955), Heilman (U.S. Pat. No. 4,270,549) and others achieved success at terminating singular events of cardiac arrhythmias on an as presented basis. The work by Diaz in provisional application, Ser. No. 60/451,005, filed Mar. 3, 2003 and utility application Ser. No. 10/757,948, filed Jan. 14, 2004, have as an aim to teach configurations, methods and apparatus ideally suited for coupling of electrical energy to the heart muscle efficiently enough and with proper catheter/lead design that not only low and medium energy (0.5 to 30 Joules) but also high energy (over 30 Joules) could be coupled safely.

To test these concepts on an animal model, a good candidate had to be found with biological (natural) atrial fibrillation. One professional from the field was seeking a solution to the treatment of equine atrial fibrillation as an alternate or adjunctive treatment to drugs. These efforts lead to contact with one of the leading companies of such devices, Rhythm Technologies, Inc. The collaboration then expanded to include a company doing much of the development work for the Rhythm Technology devices, Polymer Component Services, Inc. or PolyComp (now Cardiac Output Technologies, Inc.). Over the last few years, a great deal of effort has been expended in an attempt to reduce theory to clinical practice. As a result, the parties have had to solve problems that are unique to equine medicine. For example, it was necessary to properly suit the catheter and electrodes to treat an equine animal. However, certain aspects of the procedure were so different that they have led to new and stand alone discovery and inventions particularly in the area of catheter guidance and also physiological monitoring during the procedure for safety and patient care.

The use of ultrasound devices to view inside of an object and or mammal is not a new concept. In U.S. Pat. No. 6,520,916, Brennen teaches that an ultra sound image can be captured from an indwelling device if the device is equipped with a vibrating stylet or mandrel. A Doppler image of the device can be recorded and used to locate device housing the vibrating stylet. This and other work claims to facilitate the imaging of devices within an animal as taught by King (U.S. Pat. No. 4,100,916), that work being originally pioneered by Rocha (U.S. Pat. No. 3,780,572), Glover (U.S. Pat. No. 4,075,883) and Heyser (U.S. Pat. No. 4,078,232). Additional relevant work by Daniels (U.S. Pat. No. 4,290,432) teaches that bubbles are sufficiently different in density from tissue that detection can be observed and measurements made. Johnson (U.S. Pat. No. 3,710,615) also teaches the ability to measure varying densities within liquids and solids and discern, for example, the concentration of oil in water.

It therefore appears to be understood in the prior art that ultrasound can be used in soft tissue and that when a dense material is used within that tissue an image can be adequately collected and presented; such as a surgical instruments. However, when one is trying to use flexible medical devices, primarily made of elastomers or plastics within the body, a set of problems are encountered. The relative density differences are smaller and therefore the image is poor. Varying types of tissue such as bone, cartilage and fluid/air filled organs such the lungs can further complicate imaging deep within the body. In order for a device to be used successfully, one must employ more than just the ultrasound image to guide the device into place. In fact the indwelling device itself must provide some feedback so that the ultrasound image can be utilized more effectively as a guidance instrument. The present invention builds upon the teachings of Rocha, King, and Johnson to preferentially design the devices with features that are conducive to ultrasound image enhancement and that are useful, in part, in navigating devices to specific locations.

### SUMMARY OF THE INVENTION

The present invention teaches the proper anatomical positioning of catheters used to treat equine atrial fibrillation. In the preferred form of the invention, a device and method are provided for “steering” and “guiding” one or more apparatuses deep into the body and more specifically into the heart. The method described is a multiple indicator/feedback approach to the placement of the devices and confirmation of proper placement using ultrasound, pressure gradients and depth measurements to guide devices to specific target locations.

The method is ideally suited to place the device into regions in and around the heart of a large mammal such as an equine animal and, more specifically, into the heart and anatomically linked body lumens such as the pulmonary artery and pulmonary veins for a variety of clinically relevant reasons. One preferred embodiment is the termination of atrial fibrillation using high energy defibrillation.

The present invention provides a series of steps and elements of a device which will allow catheters to be navigated into the body lumens and cavities and more specifically, the heart. The measurements are taken from a set of indwelling and external measurements used to monitor the anatomical location of device treatment section so as to provide a simple non x-ray based guidance system, that can be a manual or automatic system, referred to as a quasi-visual-sensory-algorithm (Q-VSA).

In the method of equine cardioversion of the present invention a catheter is inserted into the jugular vein of horse and then mechanically guided into the heart by way of mechanical displacement monitoring. At the same time, device based pressure gradient measurements are used to confirm placement. Additional ultrasound imaging can be done to ensure proper location and placement of the catheter. In the next step of the method, device based electrical cardioversion is then performed.

### BRIEF DESCRIPTION OF THE DRAWINGS

FIG. 1 shows a preferred embodiment of catheter placement for the treatment of equine atrial fibrillation using a two catheter system with each catheter having a single high surface electrode.

FIG. 2 shows a preferred embodiment of a catheter fashioned with a pressure gradient lumen distal to a high surface electrode and just proximal of the distal tip.

FIG. 3 shows a preferred embodiment of a catheter fashioned with a dual pressure gradient lumen set-up so that one lumen is located distal to a high surface electrode, the other lumen being located proximal to high surface electrode.

FIGS. 4 and 4B shows a preferred embodiment of a catheter of the invention fashioned with an ultrasound enhancing ring located proximal to the high surface electrode for purposes of enhancing the ultrasound image.

FIG. 5 shows a the distal tip of the catheter made of high density metal and preferably alloy of Noble metal and further equipped with ultrasound reflective adhesive ring or bond joint affixing the distal tip to the catheter body; the design serving to enhance the ultrasound image and facilitate an additional or alternate inter-cavitary image system, such as a echosonograph.

FIGS. 6A and 6B shows the distal end of the catheter fashioned with an orientation marker designed to provide feedback of curve orientation and/or amount of deflection, if using active deflection, or catheter distal end when using ultrasound of echosonograph as guidance system.

FIGS. 7A-7C shows an image of pressure gradient taken from an indwelling device that is used to guide the catheter into the right atrium by use of mechanical displacement measurements and device based indwelling pressure gradient monitoring.

FIGS. 8A-8D shows an image of pressure gradient taken from an indwelling device that is used to guide the catheter into the right atrium by use of mechanical displacement measurements and device based indwelling pressure gradient monitoring ultrasound then being used to ensure placement of device occurs into left pulmonary artery.

### DETAILED DESCRIPTION OF THE INVENTION

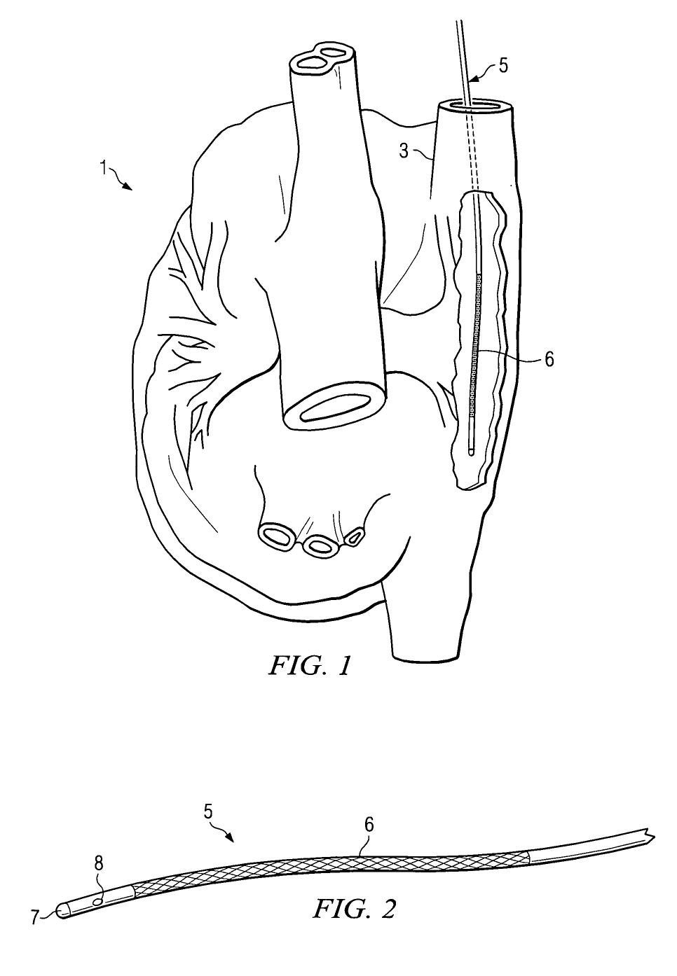

The present invention teaches the proper anatomical positioning of catheters, which, in the preferred form, are used to treat equine atrial fibrillation. FIG. 1 shows an image of the proper placement of catheters within the equine heart 1. A catheter 5 is placed in the right atrium 3 and said catheter equipped with a high surface electrode 6 (see FIG. 2) that acts as a cathode/anode. A second catheter is 5 is advanced into pulmonary artery 4 and more specifically the left pulmonary branch using the same mechanical and device based pressure gradients guidance techniques, the catheter being equipped with a high surface electrode 6, which acts as a cathode/anode.

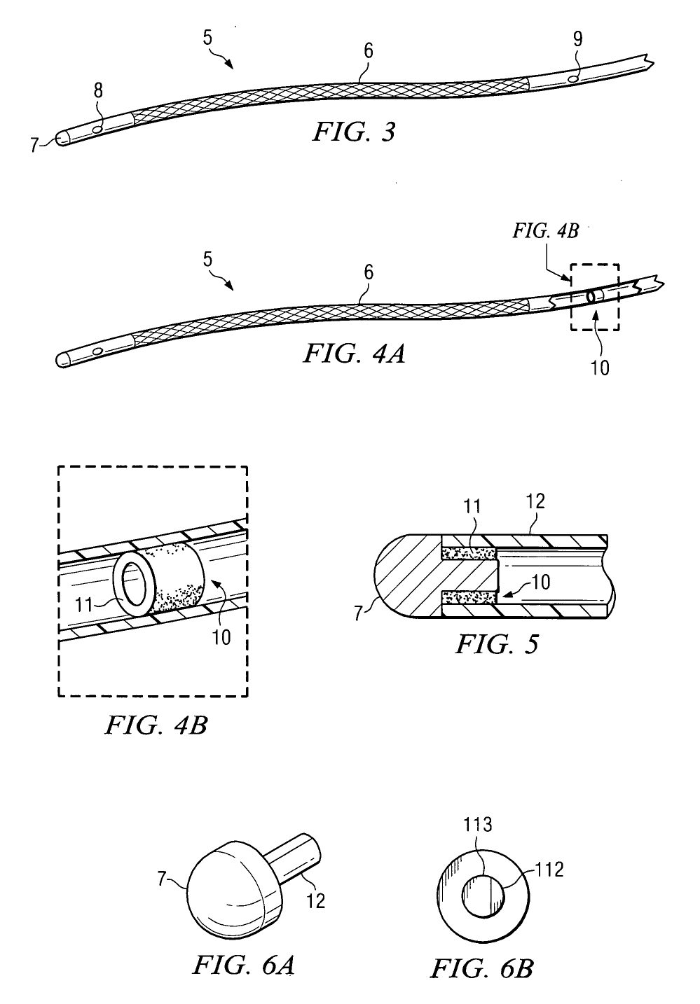

The devices are designed to facilitate the technique and include several specific design attributes. FIG. 2 shows one preferred embodiment of the present invention in which a catheter 5 is equipped with a pressure sensing means 8 and an a traumatic distal tip 7 that is visible by using ultrasound or X-ray and also high surface electrode 6. The catheter is otherwise designed with normal design attributes known in the art to enhance handling and guidance by use of braided, torqueable body. An additional preferred embodiment of the present invention is the use of two catheter based pressure sensing means. FIG. 3 shows a catheter equipped with two pressure sensing means 8, 9. One sensor 9, is located distal to the high surface electrode and another sensing port 8 is located proximal to the high surface electrode 6. The dual sensor design allows the use of pressure gradients to improve placement of the catheter into the pulmonary artery or other body lumen or organ(s) separated by valve(s). The first sensor 8 disposed in front of high surface electrode 6 senses the leading edge of catheter environment. The second sensor 9 behind the high surface electrode senses the environment behind the high surface electrode 6. One preferred embodiment of the present invention is the use of the dual pressure sensing design to help navigate a catheter into the pulmonary artery.

The devices can be ultrasound/echosonograph enhanced, so that visualization is easier, by using sound reflective markers 10, as shown in FIG. 4. The marker 10 is a composite structure of rigid or flexible plastic, epoxy or other adhesive that is used to bind together particles made of glass, ceramic, metal or clay and are geometrically ideally suited for sound reflection. The marker 10 is equipped with the composite structure 11 (see FIG. 4B) installed at strategic locations along catheter. The use of a composite structure marker 10 is also adaptable for use as a combination component for the catheter assembly. FIG. 5 shows one possible embodiment is the use of adhesives to bind the reflective material but also for the use of bonding catheter components together. The metallic distal tip 7 is bonded to the elastomeric or plastic catheter body 12 using a composite material composed of items 10 and 11 with item 11 being a bonding adhesive.

The devices can also be made so that directional orientation can be optimized using a composite material and specially machined metal parts. FIGS. 6A and 6B show one possible embodiment where the component being enhanced is the distal tip 7 of catheter. In this version of the invention the stem is cut so that the metal it is fabricated from includes a “D” shape (113 in FIG. 6B). The stem is then completed to its intended design, a column, by using non-sound reflective material 112, such as, but not limited to, plastic or epoxy. The finished component will reflect an image that has a two distinct plane differentiation based upon the fact that in one plane the stem is seen as round and in another plane the “D” shape makes the image asymmetrical. The addition can also be made on the distal or any other portion of catheter where orientation is important.

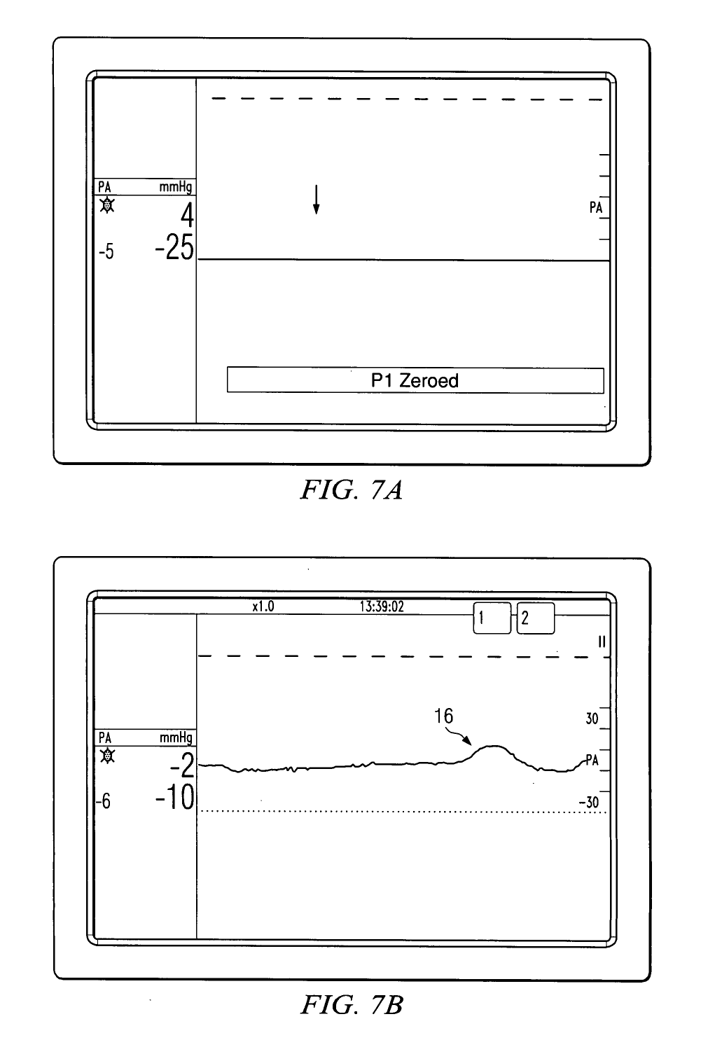

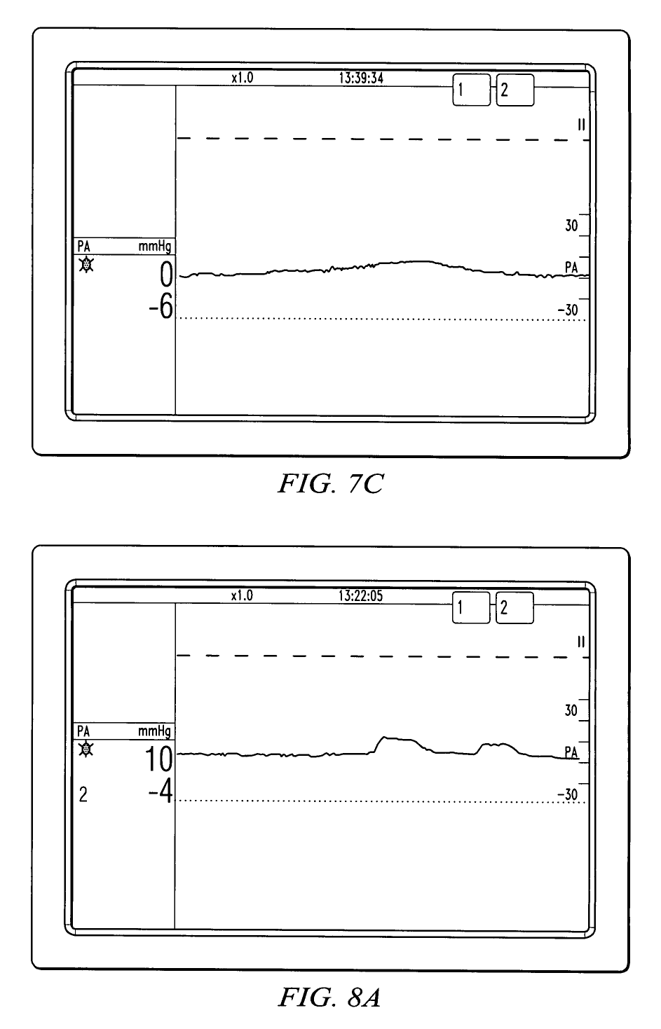

The use of the method and preferred device will now be described. A catheter equipped with sound enhancing components as taught above and catheter based pressure sensors and mechanical displacement markers or measuring system and in some cases ultrasound images is used to form a quasi-visual-sensory-algorithm (Q-VSA). FIGS. 7A-7E shows the images of an actual equine case being performed. The equine atrial fibrillation treatment process is done in three steps consisting of placing catheter into right atrium (RA) then placing a second catheter into pulmonary artery (PA) and finally delivering electrical energy. The process is started by insertion of the first catheter, the RA catheter, into jugular vein of equine and the catheter advanced about 20 centimeters with the curved section of catheter pre-disposed so that it faces inward. The catheter mounted pressure sensor is then zeroed (FIG. 7A) to the environment since absolute pressure measurements are not required but instead pressure change (gradients) are used.

The catheter is then advanced with care taken so the catheter does not twist during insertion so the curve section remains pre-disposed toward the inside of the heart. The catheter will move into the right atrium and then the curved stored energy will cause the catheters distal end to facilitate the advancement of catheter into right ventricle. The use of mechanical displacement markers and or measurement will be used to monitor advancement. The catheter mounted pressure sensor at the distal end of catheter will provide internal (indwelling) sensory information (FIG. 7B) showing when the catheter is within the right ventricle. The pressure gradient, shown in FIG. 7B, indicates the catheter distal end has entered the ventricle. The placement of the RA catheter is completed by simply pulling back the RA catheter until the pressure gradient (16 in FIG. 7B) shown disappears (see FIG. 7C) which indicates catheter distal end and high surface electrode is within the right atrium.

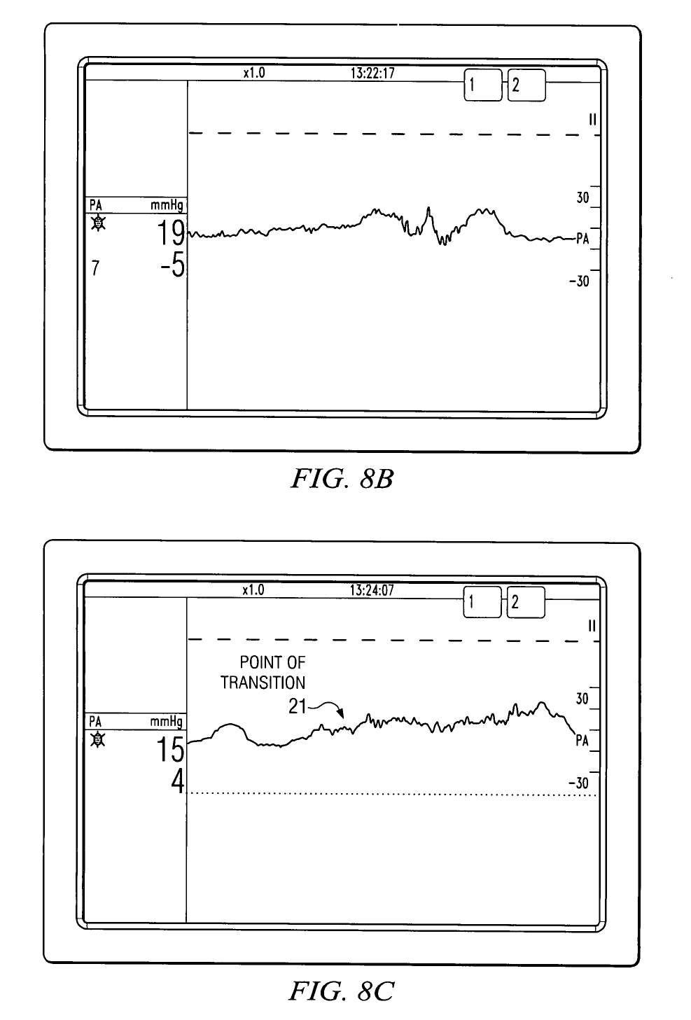

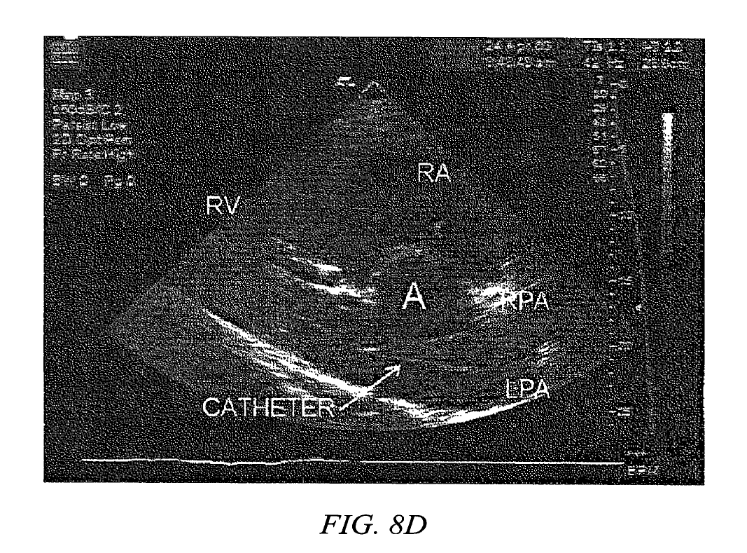

The method preferentially allows the high surface electrode to rest along the upper and latter walls of the right atrium since the stored energy of the catheter distal end will create outward mechanical force, pushing the catheter against the heart muscle. The RA catheter therefore rests against the lateral free wall of right atrium and also against the upper atrial septum. The PA catheter is then inserted in similar fashion to the RA catheter, jugular vein. The catheter is then advanced into the right ventricle as shown in FIGS. 7A-7C and both mechanical displacement and pressure gradients (see FIG. 8A) are used to confirm status. The catheter is then further advanced into the pulmonary artery outflow tract (FIG. 8B) and finally into the PA with confirmation of placement made using pressure gradient change (FIG. 8C). The transition of catheter from Right Ventricle to PA is obvious when observed using the pressure gradient 21.

The catheter is then further advanced into left pulmonary branch using ultrasound (see FIG. 8D) as the primary guidance system. The catheter is manipulated by use of torqueable body or deflectable distal end into the left pulmonary branch so that both the left and right atrial muscle mass are captured with the shock vector. The process herein disclosed is further enhanced by the use of the dual pressure system shown in FIG. 3, because the second pressure sensor 9 mounted on the catheter provides confirmation of the location of proximal end of the high surface electrode 6 in both the RA and PA to insure that catheter is ideally positioned prior to cardioversion. The second sensor would ideally be used to ensure that the PA catheter is fully inside the pulmonary artery and past the heart valve so that no ventricular mass is affected and confirm stability of catheter placements during treatment.

An invention has been provided with several advantages. The present invention teaches the use of several internal and external based measurements and ultrasound images that can be used to navigate catheters deep into the heart. The measurements are used together to create a quasi-visual-sensory-algorithm (Q-VSA). The system relies on several inputs provided to a clinician that originate from both external and internal sources. The external source is an ultrasound system image of the anatomy displayed as a cross sectional view. The internal input comprises pressure intra-luminal pressure gradients taken at or near the distal tip of catheter and an optional second catheter based input being electrical signals taken at or near the tip.

While the invention has been shown in only one of its forms, it is not thus limited but is susceptible to various changes and modifications without departing from the spirit thereof.

What is claimed is:

1. A method of guiding a catheter within a mammalian body, the method comprising the steps of:
positioning a catheter into a deep body lumen or organ using an external ultrasound image as a principle guidance system but also in conjunction with pressure gradients taken from an associated internally located device having sensors or pressure lumens mounted thereon;
using the ultrasound image and pressure gradients to guide the catheter into a desired position within the body.

2. The method of claim 1, wherein the ultrasound image which is used to guide the catheter into a specific location within the body uses anatomical distinctions and guidance decisions made from specific physiological measurements made by way of the catheter based sensors.

3. The method of claim 2, further comprising the use of multi-plane ultra sound scans done using external means and pressure gradient measurements and depth markers and depth sensors mounted on the device to form a quasi-visual-sensory-algorithm (Q-VSA);

4. The method of claim 3, wherein the algorithm of depth, image and pressure gradients is used to create an expected series of measurements that provide sufficient affirmation for placement of devices within targeted anatomical locations within the body.

5. The method of claim 4, wherein a set of indwelling devices, working in tandem, are used to generate an electric field between electrodes mounted on the two devices, the devices being guided and preferentially positioned within the heart using the Q-VSA method.

6. The method of claim 5, wherein the mammalian heart is human having two sets of chambers consisting a left and right atrium and left and right ventricles.

7. The method of claim 6, wherein the mammalian heart is an equine animal.

8. The method of claim 6, wherein the mammalian heart is a canine animal.

9. The method of claim 6, wherein the mammalian heart is a feline animal.

10. The method of claim 6, wherein the mammalian heart is a bovine animal.

11. The method of claim 6, wherein the mammalian heart is a porcine animal

12. The method of claim 6, wherein the mammalian heart is a mastodon animal

13. The method of claim 6, wherein the mammalian heart is that of mammal weighing greater than about 1 kilogram.

14. The device of claim 6, wherein the Q-VSA algorithm is obtained from a device in the form of a system which is constructed into a single transportable unit that can be field ready such that veterinary medicine and military applications can be facilitated, thereby overcoming shortcomings of more cumbersome X-ray based imaging systems.

15. The device of claim 14, wherein the indwelling device is equipped with a semi-flexible high surface electrode capable of withstanding extreme high energy electrical discharges so that large mammalian hearts, such as equine heart, can be defibrillated without thermal injury.

16. The device of claim 15, wherein the indwelling device is capable of being oriented by mechanical deformation at a specific location along its length and further equipped with ultrasound/echosonograph image enhancing attributes such as bonding adhesives or additional cast markers that contain hollow or solid micro spheres made of glass, ceramic, plastic, metal or clay and are ideally suited to reflect sound waves.

17. The method of claim 16, wherein the mechanical deformation occurs at or beyond the distal ¼ section of device and the mechanically active section is further equipped with a flexible ultrasound enhancing sub-system, the sub-system being contained within the catheter and being flexible enough to also deform.

18. The method of claim 17, wherein the mechanical deformation occurs before the device is inserted into patients such that a pre-set curve on distal end of catheter is malleable and can be adjusted prior to insertion.

19. The method of claim 18, wherein the device is fashioned with a lumen hole located on the side of catheter, the lumen being coupled to an isolated lumen and positioned between 1 mm to 50 mm from distal end of catheter.

20. The method of claim 19, wherein the device is fashioned with two lumen holes on the side and/or tip of device but located to capture or frame the high surface electrode, one of the lumen holes being at least 1 mm distal of high surface electrode and the second lumen hole being at least 1 mm proximal to high surface electrode.

21. The method of claim 20, wherein the lumen holes are coupled to independent lumen conduits for hydraulic circuit isolation.

22. The method of claim 21, wherein a selected lumen hole is coupled to a common conduit and an average pressure gradient between lumens is observed.

23. The method of claim 22, wherein the selected lumen hole is coupled to a common conduit that can be made selectively active to either lumen hole by the use of a telescoping tube that can either open or close either lumen by either blocking or allowing to stay open one, none or both of the lumen holes.

24. The method of claim 23, whereby the conduits connecting distal end lumen holes are terminated at a proximal end of the device by way of a Leur Lock™, or similar fitting.

25. The method of claim 24, wherein depth markers are applied to the circumference of the catheter in 25 centimeter increments and each thin line demarcates a 25 mm displacement, each thick line demarcates a 50 cm displacement, and a line that indicates a location where a curve arc faces inward, so that the user can see and use the mark for additional guidance.

26. The method of claim 25, wherein the devices are packaged as a set of catheters to be used in a single patient and inclusive of valves, suture straps, introducers and other sterile materials required to treat a patient, so that ease of use is achieved.

27. The method of claim 26, wherein the high energy electrodes measure 10 cm or more and are of the size no greater than 24 French (8 mm).

28. The method of claim 27, wherein the devices are equipped with a single built-in cable that connects both high energy electrodes on the two catheters by means of a single one piece connector that is customized and fashioned to connect to defibrillators readily found in the field.

29. The method of claim 28, wherein the devices are equipped with a single built in cable that connects both distal tips of the catheters and the low energy electrode on the catheters onto a single one piece connector that is customized and fashioned to connect to defibrillators that are readily found in the field.
